(19) United States
(12) Patent Application Publication
(10) Pub. No.: US 2026/0363753 A1
(43) Pub. Date: Nov. 19, 2026

(54) PULSED ELECTRIC FIELD ABLATION CATHETER AND COOLING METHOD

(71) Applicant: Shenzhen Pulsecare Medical Technology Co., Ltd., Shenzhen (CN)

(72) Inventor: Jianwen Tan, Shenzhen (CN)

(21) Appl. No.: 19/724,985

(22) Filed: Jun. 30, 2026

Related U.S. Application Data
(63) Continuation of application No. PCT/CN2025/095016, filed on May 15, 2025.

(30) Foreign Application Priority Data
May 31, 2024 (CN) 202410705660.3

(51) Int. Cl. A61B 18/00

(52) U.S. Cl. CPC A61B 18/00; A61B 2018/00011; A61B 2018/0016; A61B 2018/00184; A61B 2018/00267; A61B 2018/00577; A61B 2218/002

(57) ABSTRACT

A pulsed electric field ablation catheter includes a catheter body and an expandable portion. The catheter body includes a main tube and a push-pull tube slidably received in the main tube and extending at least partially out of a distal end of the main tube. The expandable portion includes an expandable tube and a plurality of branch tubes formed between proximal and distal ends of the expandable tube. At least one therapeutic electrode is disposed on each branch tube, and a cooling gap is defined between the branch tube and the therapeutic electrode and in communication with an external environment. A first irrigation lumen is provided in the main tube, and a second irrigation lumen is provided in each branch tube. The first irrigation lumen is in fluid communication with the second irrigation lumen, and the second irrigation lumen is in fluid communication with the cooling gap.

### CROSS-REFERENCE TO RELATED APPLICATIONS

The present application is a Continuation Application of PCT Application No. PCT/CN2025/095016, filed on May 15, 2025, which claims priority of Chinese Patent Application No. 202410705660.3, filed on May 31, 2024, the entire contents of which are hereby incorporated by reference.

### TECHNICAL FIELD

The present application relates to the technical field of medical devices, and more particularly to a pulsed electric field ablation catheter and a cooling method.

### BACKGROUND

A pulsed electric field ablation catheter is a medical device used for cardiac electrophysiological ablation procedures. The working principle of the pulsed electric field ablation catheter is to utilize the non-thermal effect of irreversible electroporation (IRE) to deliver high-voltage pulse energy into the heart via catheter electrodes, thereby causing irreversible electroporation of myocardial cell membranes and subsequent necrosis of myocardial cells, so as to block the propagation of abnormal electrical signals and treat arrhythmia.

### SUMMARY

In some embodiments, the present disclosure provides a pulsed electric field ablation catheter comprising: a catheter body and an expandable portion, the catheter body comprising a main tube and a push-pull tube being slidably received in the main tube and extending at least partially out of a distal end of the main tube, the expandable portion comprising an expandable tube and a plurality of branch tubes formed between a proximal and a distal end of the expandable tube, the plurality of branch tubes being circumferentially spaced apart along the expandable tube, the proximal end of the expandable tube being connected to the distal end of the main tube, and the distal end of the expandable tube being connected to a distal end of the push-pull tube; at least one therapeutic electrode being disposed on each branch tube, and a cooling gap being defined between the branch tube and the therapeutic electrode, the cooling gap being in communication with an external environment; a first irrigation lumen being provided in the main tube, and a second irrigation lumen being provided in each branch tube, the first irrigation lumen being in fluid communication with the second irrigation lumen, and the second irrigation lumen being in fluid communication with the cooling gap.

In some embodiments, the present disclosure provides a cooling method applied to a pulsed electric field ablation catheter, the pulsed electric field ablation catheter comprising a catheter body and an expandable portion, the catheter body comprising a main tube and a push-pull tube being slidably received in the main tube and extending at least partially out of a distal end of the main tube; the expandable portion comprising an expandable tube and a plurality of branch tubes formed between a proximal and a distal end of the expandable tube, the proximal end of the expandable tube being connected to the distal end of the main tube, and the distal end of the expandable tube being connected to a distal end of the push-pull tube; at least one therapeutic electrode being disposed on each branch tube, and a cooling gap being defined between the branch tube and the therapeutic electrode, the cooling gap being in communication with an external environment; a first irrigation lumen being provided in the main tube, and a second irrigation lumen being provided in each branch tube, the first irrigation lumen being in fluid communication with the second irrigation lumen, and the second irrigation lumen being in fluid communication with the cooling gap; and the method comprising: receiving an irrigation fluid via the first irrigation lumen; delivering the irrigation fluid from the first irrigation lumen to the second irrigation lumen; and delivering the irrigation fluid from the second irrigation lumen to the cooling gap.

### BRIEF DESCRIPTION OF THE DRAWINGS

For a clearer description of the technical solutions of the embodiments of the present disclosure, the drawings required for the description of the embodiments or the prior art are briefly described below. It should be understood that the drawings described below illustrate only some embodiments of the present disclosure, and other drawings may be derived from these drawings by those skilled in the art without creative effort.

FIG. 1 is a schematic structural view of a pulsed electric field ablation catheter according to at least one embodiment of the present disclosure.

FIG. 2 is a partial cross-sectional view of a pulsed electric field ablation catheter according to at least one embodiment of the present disclosure.

FIG. 3 is another partial cross-sectional view of a pulsed electric field ablation catheter according to at least one embodiment of the present disclosure.

FIG. 4 is yet another partial cross-sectional view of a pulsed electric field ablation catheter according to at least one embodiment of the present disclosure.

FIG. 5 is a partial schematic working view of a pulsed electric field ablation catheter according to at least an embodiment of the present disclosure, in which no cold saline is delivered to cool the therapeutic electrode.

FIG. 6 is a partial schematic working view of a pulsed electric field ablation catheter according to at least an embodiment of the present disclosure, in which cold saline is delivered to cool the therapeutic electrode.

FIG. 7 is a partial cross-sectional view of a pulsed electric field ablation catheter according to at least an embodiment of the present disclosure, in which a tip is enveloped by cold saline.

FIG. 8 is a flowchart of a cooling method according to at least one embodiment of the present disclosure.

FIG. 9 is a flowchart of another cooling method according to at least one embodiment of the present disclosure.

Reference numerals in the above drawings are listed as follows:
101, proximal end; 102, distal end; 103, flow direction of cold saline;
10, catheter body; 11, main tube; 111, first irrigation lumen; 112, push-pull channel; 12, push-pull tube; 13, connecting tube;
20, expandable portion; 21, expandable tube; 211, branch tube; 2111, second irrigation lumen; 2112, irrigation hole; 212, therapeutic electrode; 22, first sealing member; 23, second sealing member;
30, cooling gap; 31, cooling recess;
40, distribution chamber;
50, power supply line;
60, blood;
70, mixed conductive solution;
80, cold saline;
90, tip.

### DESCRIPTION OF THE EMBODIMENTS

To make the technical problems to be solved, the technical solutions, and the beneficial effects of the present disclosure clearer, the present disclosure will be further described in detail below with reference to the accompanying drawings and embodiments. It should be understood that the specific embodiments described herein are merely illustrative and are not intended to limit the present disclosure.

It should be noted that when an element is referred to as “fixed to” or “disposed on” another element, it may be directly on the other element or indirectly on the other element with one or more intervening elements therebetween. When an element is referred to as being “connected to” another element, it may be directly connected to the other element or indirectly connected through intervening elements. Provided there is no conflict, the embodiments and features of the present disclosure can be combined with each other. The present disclosure will be described in detail below with reference to the accompanying drawings and embodiments.

It should be understood that terms such as “length”, “width”, “upper”, “lower”, “front”, “back”, “left”, “right”, “vertical”, “horizontal”, “top”, “bottom”, “inner”, “outer”, and the like refer to the orientations or positional relationships shown in the accompanying drawings and are used merely for the convenience of describing and simplifying the present disclosure, rather than indicating or implying that the referenced device or element must have a particular orientation or be constructed and operated in a particular orientation. Therefore, they should not be construed as limiting the present disclosure.

Moreover, the terms “first” and “second” are used for descriptive purposes only and are not to be construed as indicating or implying relative importance or implicitly indicating the number of the indicated technical features. Thus, a feature defined by “first” and “second” may explicitly or implicitly includes one or more of such features. As used herein, “multiple” means two or more, unless expressly specified otherwise.

A pulsed electric field ablation catheter is a medical device used for cardiac electrophysiological ablation procedures. The working principle of the pulsed electric field ablation catheter is to deliver a high-intensity pulsed electric field to cardiac tissue through the catheter to disrupt abnormal cardiac conduction pathways, thereby treating arrhythmias and other conditions. The pulsed electric field ablation catheter generally includes a catheter body, one or more electrodes, and a pulse generator. During a procedure, the catheter is advanced through a blood vessel into the heart to place the electrodes in contact with a target ablation site. The pulse generator is then activated to generate a pulsed electric field that induces irreversible electroporation and apoptosis of cells, thereby achieving ablation. During operation, the electrodes deliver high-voltage pulsed energy. When the electrodes are closely spaced, spark discharge may occur in the blood, generating localized high temperatures that can cause thrombus or eschar formation. Therefore, it is necessary to control the temperature of the electrodes in real time.

In existing pulsed electric field ablation catheters, electrode temperature rise is generally controlled by cold saline irrigation. To provide cold saline irrigation to the electrodes, an irrigation lumen is provided in the catheter body, and irrigation holes are formed in surfaces of the electrodes. The cold saline is delivered to the electrodes through the irrigation holes to control the electrode temperature. However, forming irrigation holes in the electrode surfaces may lead to the formation of sharp edges or points. When the sharp edges or points are present on the electrode surfaces, there is a risk of spark discharge near the sharp edges or points during operation, which can cause localized high temperatures and potentially result in thrombus or eschar formation, compromising the efficacy and safety of the procedure.

As used herein, the term “electroporation” refers to applying an electric field to a cell membrane to alter its permeability of the cell membrane to the extracellular environment, and the term “irreversible electroporation” refers to applying an electric field to a cell membrane to permanently alter its permeability of the cell membrane to the extracellular environment. For example, in cells undergoing irreversible electroporation, one or more pores can be observed in the cell membrane, and these pores remain after the electric field is removed. As used herein, the term “proximal end” (labeled 101) refers to an end of the pulsed electric field ablation catheter that is close to the operator or the end that is connected to the operator, and the term “distal end” (labeled 102) refers to an end of the pulsed electric field ablation catheter that is inserted into the blood vessel or the end that is close to the tissue to be treated.

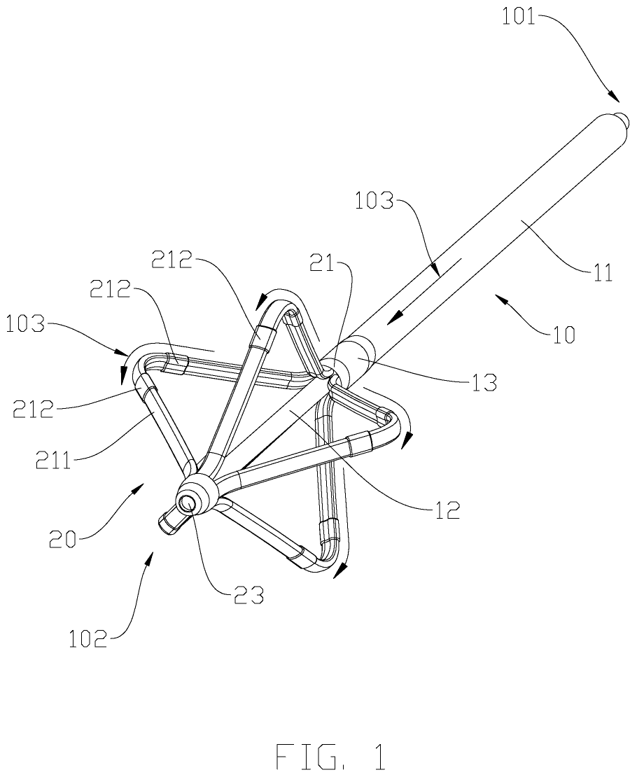

Referring to FIGS. 1-7, in order to solve at least one of the above-mentioned problems, according to one aspect of the present disclosure, at least one embodiment of the present disclosure provides a pulsed electric field ablation catheter, which includes a catheter body 10 and an expandable portion 20. The catheter body 10 includes a main tube 11 and a push-pull tube 12 slidably received in the main tube 11 and extending at least partially out of a distal end of the main tube 11. The expandable portion 20 includes an expandable tube 21 and a plurality of branch tubes 211 formed between a proximal end and a distal end of the expandable tube 21.

For example, the plurality of branch tubes 211 are circumferentially spaced apart along the expandable tube 21.

The proximal end of the expandable tube 21 is connected to the distal end of the main tube 11, and the distal end of the expandable tube 21 is connected to the distal end of the push-pull tube 12.

At least one therapeutic electrode 212 is disposed on each branch tube 211, and a cooling gap 30 is defined between the branch tube 211 and the therapeutic electrode 212, the cooling gap 30 being in communication with an external environment. The main tube 11 is provided with a first irrigation lumen 111, and the branch tube 211 is provided with a second irrigation lumen 2111. The first irrigation lumen 111 is in fluid communication with the second irrigation lumen 2111, and the second irrigation lumen 2111 is in fluid communication with the cooling gap 30.

In the pulsed electric field ablation catheter provided by at least one embodiment of the present disclosure, the push-pull tube 12 is slidably received in the main tube 11 and extends at least partially out of the distal end of the main tube 11. The expandable tube 21 has its proximal end connected to the distal end of the main tube 11 and its distal end connected to the distal end of the push-pull tube 12. Thus, the plurality of branch tubes 211 located between the proximal and distal ends of the expandable tube 21 are capable of deforming in a radial direction of the expandable tube 21 along with the movement of the push-pull tube 12. By disposing at least one therapeutic electrode 212 on each branch tube 211, the position of the therapeutic electrode 212 in the radial direction of the expandable tube 21 is adjustable along with the deformation of the branch tube 211. Meanwhile, a cooling gap 30 is defined between the branch tube 211 and the therapeutic electrode 212, the cooling gap 30 being in communication with an external environment. The main tube 11 is provided with a first irrigation lumen 111, and the branch tube 211 is provided with a second irrigation lumen 2111. The first irrigation lumen 111 is in fluid communication with the second irrigation lumen 2111, and the second irrigation lumen 2111 is in fluid communication with the cooling gap 30. Accordingly, an irrigation fluid (such as cold saline 80) in the first irrigation lumen 111 can flow through the second irrigation lumen 2111 into the cooling gap 30 between the branch tube 211 and the therapeutic electrode 212, and then circulate to the external environment, thereby achieving cooling of the therapeutic electrode 212. As shown in FIGS. 1-7, a flow direction 103 of cold saline is shown by arrows.

The pulsed electric field ablation catheter provided by at least one embodiment of the present disclosure eliminates the need to form irrigation holes 2112 in the surface of the therapeutic electrode 212, which helps avoid spark discharge on the therapeutic electrode 212, thereby achieving high reliability.

During ablation of target tissue using the pulsed electric field ablation catheter provided by at least one embodiment of the present disclosure, the irrigation fluid (such as cold saline 80) can be discharged to the vicinity of the therapeutic electrode 212 through the cooling gap 30 and mixed with blood near the therapeutic electrode 212, thereby reducing the impedance of the therapeutic electrode 212 and allowing the pulsed electric field generated on the therapeutic electrode 212 to act on the target tissues more easily. In addition, when a tip 90 is present on the therapeutic electrode 212, the cold saline 80 discharged from the cooling gap 30 to the vicinity of the therapeutic electrode 212 can also envelop the tip 90 in the form of water droplet, thereby improving the uniformity of the electric field distribution on an end face of the therapeutic electrode 212 and the tip 90, and preventing spark discharge caused by uneven electric field distribution of the therapeutic electrode 212.

Referring to FIG. 1, in an exemplary embodiment, there are five branch tubes 211. It should be understood, however, that in other embodiments, the number of branch tubes 211 may be other numbers.

Referring to FIG. 3, in an exemplary embodiment, the expandable tube 21 is disposed around the push-pull tube 12. The expandable portion 20 further includes a first sealing member 22 disposed on the proximal end of the expandable tube 21 and positioned between the expandable tube 21 and the push-pull tube 12 to seal a gap between the expandable tube 21 and the push-pull tube 12.

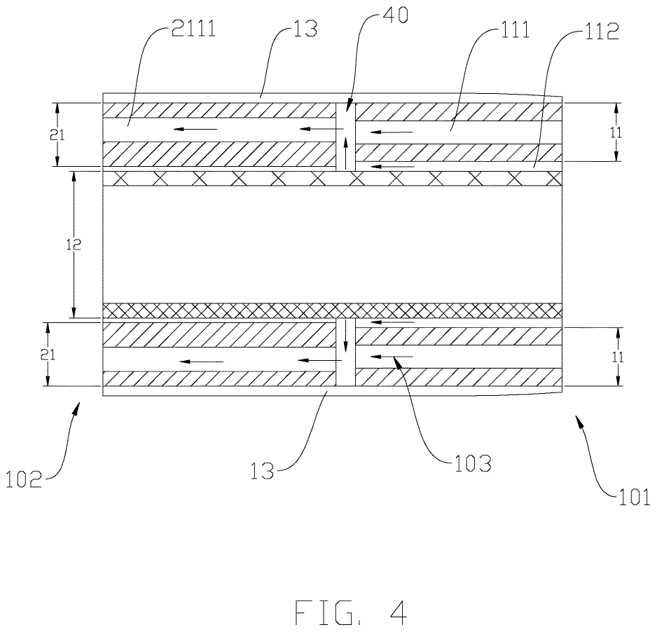

By disposing the expandable tube 21 around the push-pull tube 12, the plurality of branch tubes 211 can be positioned radially outward of the push-pull tube 12, such that the plurality of branch tubes 211 can deform in the radial direction of the expandable tube 21 along with the movement of the push-pull tube 12. At the same time, by disposing the first sealing member 22 on the proximal end of the expandable tube 21 and positioning the first sealing member 22 between the expandable tube 21 and the push-pull tube 12, the gap between the expandable tube 21 and the push-pull tube 12 can be sealed by the first sealing member 22 of the pulsed electric field ablation catheter. Referring to FIG. 1 and FIG. 4, in an exemplary embodiment, the catheter body 10 further includes a connecting tube 13. The connecting tube 13 has its two ends respectively disposed around the proximal end of the expandable tube 21 and the distal end of the main tube 11 to connect the expandable tube 21 and the main tube 11. For example, the two ends of the connecting tube 13 are respectively secured to the proximal end of the expandable tube 21 and the distal end of the main tube 11.

In an optional embodiment, the connecting tube 13 is a Pebax thin-walled tube. A distal inner wall of the Pebax thin-walled tube is thermally bonded to a proximal outer wall of the expandable tube 21, and a proximal inner wall of the Pebax thin-walled tube is thermally bonded to a distal outer wall of the main tube 11, thereby connecting and communicating the expandable tube 21 and the main tube 11.

Referring to FIG. 4, in order to uniformly distribute the cold saline from the first irrigation lumen 111 into the plurality of second irrigation lumens 2111, a distribution chamber 40 is formed among the proximal end of the expandable tube 21, the distal end of the main tube 11, and an inner wall of the connecting tube 13. The first irrigation lumen 111 is in fluid communication with the second irrigation lumens 2111 via the distribution chamber 40. Illustratively, a gap is provided between the proximal end of the expandable tube 21 and the distal end of the main tube 11, thereby defining at least a portion of the distribution chamber 40. By providing the distribution chamber 40 among the proximal end of the expandable tube 21, the distal end of the main tube 11, and the inner wall of the connecting tube 13, the first irrigation lumen 111 is placed in fluid communication with the plurality of second irrigation lumens 2111 simultaneously through the distribution chamber 40, so that the cold saline in the first irrigation lumen 111 can be uniformly distributed into the plurality of second irrigation lumens 2111.

The pulsed electric field ablation catheter generally includes a relatively large number of electrodes, and each electrode requires a corresponding irrigation lumen for cooling. However, the diameter of the main tube 11 is limited. As the number of irrigation lumens increases, the inner diameter of each individual irrigation lumen decreases, leading to lower space utilization of the main tube 11. Consequently, the flow rate of cold saline through the irrigation lumen is restricted, which may compromise the cooling effect to the electrode. In contrast, in the pulsed electric field ablation catheter provided by at least one embodiment of the present disclosure, only a single first irrigation lumen 111 is provided in the main tube 11, and is in fluid communication with the plurality of second irrigation lumens 2111 via the distribution chamber 40. Thus, there is no need to provide a corresponding number of irrigation lumens in the main tube 11, which achieves higher space utilization of the main tube 11 and effectively increases the flow rate of cold saline irrigation.

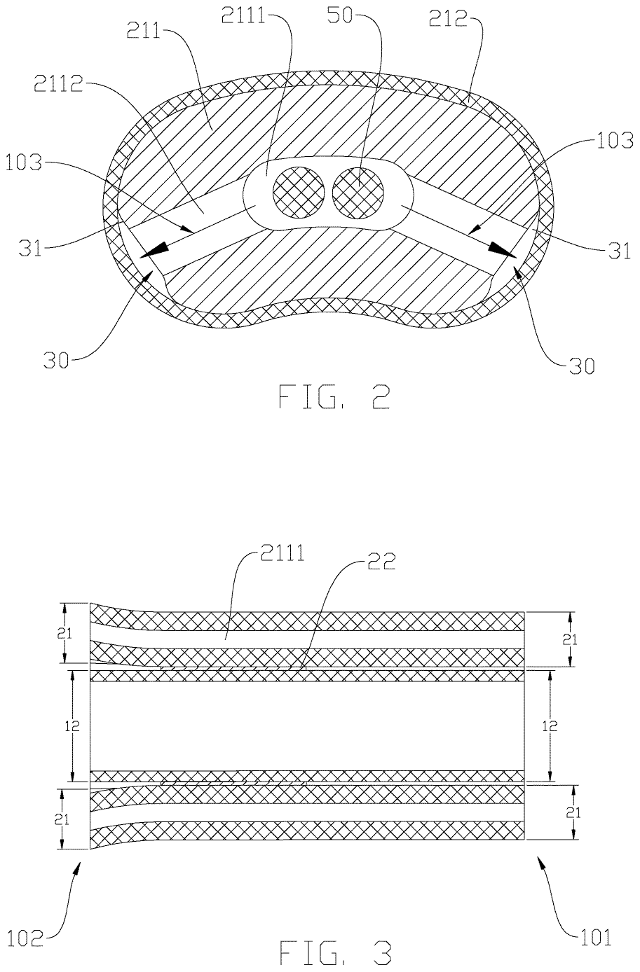

Referring to FIG. 2, in an exemplary embodiment, an irrigation hole 2112 is defined in the branch tube 211 at a position corresponding to the therapeutic electrode 212, for placing the second irrigation lumen 2111 in fluid communication with the cooling gap 30. By providing the irrigation hole 2112 in the branch tube 211 at the position corresponding to the therapeutic electrode 212, the second irrigation lumen 2111 is placed in fluid communication with the cooling gap 30 through the irrigation hole 2112 in the pulsed electric field ablation catheter.

Referring to FIG. 2, in an exemplary embodiment, a cooling recess 31 is provided on an outer wall of the branch tube 211 at a position corresponding to the therapeutic electrode 212. The cooling gap 30 is formed between the cooling recess 31 and the therapeutic electrode 212. The irrigation hole 2112 has one end in fluid communication with the cooling gap 30 formed by the cooling recess 31 and the other end in fluid communication with the second irrigation lumen 2111. Illustratively, the irrigation hole 2112 is defined in and extends through a wall of the branch tube 211, such that a length of the irrigation hole 2112 corresponds to a thickness of the wall of the branch tube 211. The two ends of the irrigation hole 2112 along its length direction are in fluid communication with the cooling recess 31 and the second irrigation lumen 2111, respectively. By providing the cooling recess 31 on the outer wall of the branch tube 211 at the position corresponding to the therapeutic electrode 212, the cooling gap 30 can be formed between the cooling recess 31 and the therapeutic electrode 212.

Referring to FIG. 1 and FIG. 2, in an exemplary embodiment, the therapeutic electrode 212 is an annular electrode mounted around the branch tube 211.

Both the cooling recess 31 and the irrigation holes 2112 are provided in plurality. The plurality of cooling recesses 31 are circumferentially spaced apart along the branch tube 211, and the plurality of irrigation holes 2112 are in one-to-one correspondence with the plurality of cooling recesses 31.

By providing a plurality of cooling recesses 31 and a plurality of irrigation holes 2112, with the plurality of cooling recesses 31 circumferentially spaced apart along the branch tube 211 and the plurality of irrigation holes 2112 in one-to-one correspondence with the plurality of cooling recesses 31, cold saline can be delivered to the electrodes through a plurality of cooling gaps 30, thereby effectively improving the cooling effect on the electrodes.

In an optional embodiment, two cooling recesses 31 and two irrigation holes 2112 are provided. The two cooling recesses 31 are respectively disposed on two sides of the branch tube 211 in the circumferential direction of the expandable tube 21, and the two irrigation holes 2112 correspond to the two cooling recesses 31, respectively. In other words, the two cooling recesses 31 are respectively disposed on two sides of the branch tube 211 along a width direction of the branch tube 211, and are distributed along the circumferential direction of the expandable tube 21.

Referring to FIG. 3, in an exemplary embodiment, the first sealing member 22 includes a sealing tube inserted into the expandable tube 21 and disposed around the push-pull tube 12. An outer wall of the sealing tube is in contact with an outer wall of the expandable tube 21, and an inner wall of the sealing tube is in contact with an outer wall of the push-pull tube 12. With the sealing tube inserted into the expandable tube 21 and disposed around the push-pull tube 12, the outer wall of the sealing tube is fixedly connected to the inner wall of the expandable tube 21, and the inner wall of the sealing tube is in contact with the outer wall of the push-pull tube 12, the gap between the inner wall of the expandable tube 21 and the outer wall of the push-pull tube 12 can be effectively sealed by the sealing tube.

In an optional embodiment, the sealing tube is a TPU thin-wall tube, and is fixedly connected to the inner wall of the expandable tube 21 by adhesive bonding.

In an optional embodiment, the TPU Thin-wall tube is in interference fit with the push-pull tube 12. Due to the elasticity of the TPU thin-wall tube, the TPU thin-walled tube functions as a dynamic seal during push-pull movement of the push-pull tube 12. This facilitates smooth push-pull operation of the push-pull tube 12 while providing real-time sealing the gap between the inner wall of the expandable tube 21 and the outer wall of the push-pull tube 12.

Referring to FIG. 1, in an exemplary embodiment, multiple therapeutic electrodes 212 are provided on the branch tube 211 and are spaced apart along a longitudinal direction of the branch tube 211. By providing multiple therapeutic electrodes 212 spaced apart along the longitudinal direction of the branch tube 211, the pulsed electric field ablation catheter can achieve improved therapeutic efficacy.

In an optional embodiment, the multiple therapeutic electrodes 212 are spaced apart and disposed on a middle portion of the branch tube 211.

In an optional embodiment, a distance between two adjacent therapeutic electrodes 212 ranges from 1.5 mm to 3 mm. For example, the distance between two adjacent therapeutic electrodes 212 is 2 mm. It should be understood that the value of 2 mm is described merely for ease of understanding the technical solution, and may vary within a range of approximately 2 mm, with a tolerance of ±0.5 mm.

In an optional embodiment, two therapeutic electrodes 212 are provided on a single branch tube 211. It should be understood that, in other embodiments, the therapeutic electrodes 212 on a single branch tube 211 may also be provided in other numbers.

Referring to FIG. 1, in an exemplary embodiment, to prevent the cold saline in the second irrigation lumen 2111 from flowing out of the distal end thereof into the external environment, the expandable portion 20 further includes a second sealing member 23. The second sealing member 23 is disposed on the distal end of the expandable tube 21 to seal the distal end of the second irrigation lumen 2111. By providing the second sealing member 23 on the distal end of the expandable tube 21, the distal end of the second irrigation lumen 2111 of the pulsed electric field ablation catheter can be sealed by the second sealing member 23. This helps prevent the cold saline in the second irrigation lumen 2111 from flowing out of the distal end thereof into the external environment, and ensures that the cold saline in the second irrigation lumen 2111 flow out through the cooling gap 30 via the irrigation holes 2112, thereby allowing the therapeutic electrode 212 to achieve sufficient cooling.

Referring to FIG. 2, in an optional embodiment, the pulsed electric field ablation catheter further includes a power supply line 50. The power supply line 50 is at least partially received in the second irrigation lumen 2111 and is electrically connected to the therapeutic electrodes 212. An external pulse power supply device can supply power to the therapeutic electrodes 212 via the power supply line 50.

In an optional embodiment, the power supply line 50 is an enameled wire.

In an optional embodiment, the main tube 11 is provided with a push-pull channel 112, and the push-pull tube 12 is slidably received in the push-pull channel 112. The push-pull channel 112 is in fluid communication with the distribution chamber 40, such that cold saline can be delivered through a gap between the push-pull tube 12 and the push-pull channel 112 into the distribution chamber 40.

Referring to FIG. 5, in an exemplary embodiment, when cold saline 80 is not being delivered to cool the therapeutic electrode 212, the pulsed electric field generated on the therapeutic electrode 212 must pass through the blood 60 to act on the target tissue, which may lead to high impedance and suboptimal treatment effect.

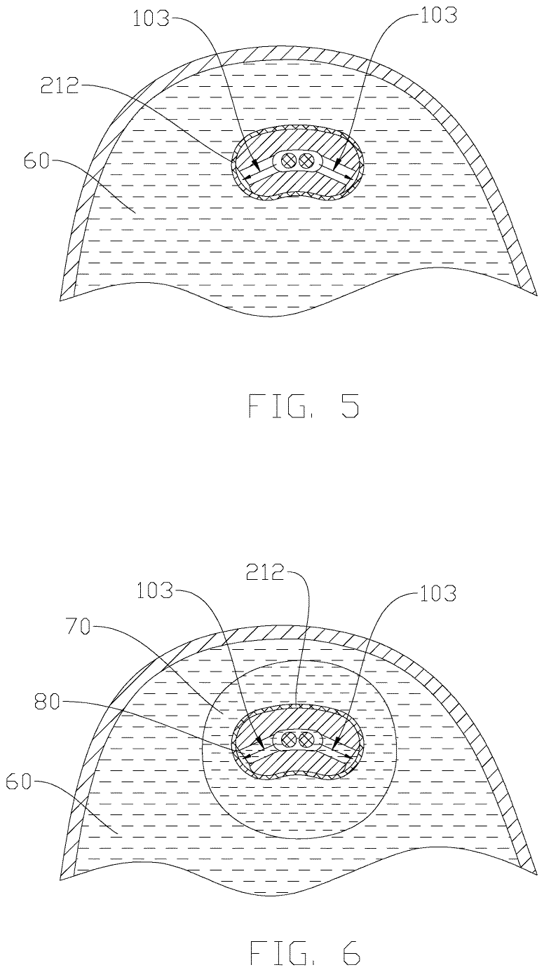

Referring to FIG. 6, in an exemplary embodiment, when the pulsed electric field ablation catheter delivers cold saline 80 to cool the therapeutic electrode 212, the cold saline 80 is discharged to the vicinity of the therapeutic electrode 212 through the cooling gap 30, increasing a contact area between the therapeutic electrode 212 and the blood 60. Further, the cold saline 80 has a certain conductivity and an impedance generally lower than that of the blood 60. Thus, the cold saline 80 can mix with the blood 60 near the therapeutic electrode 212 to form a mixed conductive solution 70, which may help reduce a treatment impedance of the therapeutic electrode 212, allowing the pulsed electric field generated on the therapeutic electrode 212 to act on the target tissue more easily, thereby improving a treatment effect of the therapeutic electrode 212.

In an exemplary embodiment, referring to FIG. 7, in the case that a tip 90 is present on the therapeutic electrode 212, the pulsed electric field ablation catheter can discharge the cold saline 80 to the vicinity of the therapeutic electrode 212 through the cooling gap 30. The cold saline 80 envelops the tip 90 in the form of water droplet, which helps improve uniformity of an electric field distribution on an end face of the therapeutic electrode 212 and the tip 90, thereby preventing spark discharge caused by uneven electric field distribution of the therapeutic electrode 212.

In an exemplary embodiment, the pulsed electric field ablation catheter provides at least the following beneficial technical effects. The push-pull tube 12 is slidably received in the main tube and extends at least partially out of the distal end of the main tube 11, and the two ends of the expandable tube 21 are connected to the distal end of the main tube 11 and the distal end of the push-pull tube 12, respectively. Thus, the plurality of branch tubes 211 located between the proximal and distal ends of the expandable tube 21 can deform in the radial direction of the expandable tube 21 along with the movement of the push-pull tube 12. Moreover, by disposing at least one therapeutic electrode 212 on the branch tube 211, the position of the therapeutic electrode 212 can be adjusted in the radial direction of the expandable tube 21 along with the deformation of the branch tube 211. At the same time, a cooling gap 30 in communication with an external environment is provided between the branch tube 211 and the therapeutic electrode 212, a first irrigation lumen 111 is provided in the main tube 11, and a second irrigation lumen 2111 is provided in the branch tube 211. The first irrigation lumen 111 is in fluid communication with the second irrigation lumen 2111, and the second irrigation lumen 2111 is in fluid communication with the cooling gap 30. Accordingly, the cold saline in the first irrigation lumen 111 can flow into the cooling gap 30 between the branch tube 211 and the therapeutic electrode 212 via the second irrigation lumen 2111 and then circulate to the external environment, thereby cooling the therapeutic electrode 212. The pulsed electric field ablation catheter eliminates the need to form irrigation hole 2112 in the surface of the therapeutic electrode 212, which helps avoid spark discharge on the therapeutic electrode 212 and thus achieves high reliability. During ablation of target tissues, the cold saline 80 can be discharged to the vicinity of the therapeutic electrode 212 through the cooling gap 30 and mixed with the blood near the therapeutic electrode 212, thereby reducing the therapeutic impedance of the therapeutic electrode 212 and allowing the pulsed electric field generated on the therapeutic electrode 212 to act on the target tissues more easily. In addition, in the case that a tip 90 is present on the therapeutic electrode 212, the cold saline 80 discharged from the cooling gap 30 to the vicinity of the therapeutic electrode 212 can envelop the tip 90 in the form of water droplet. This helps improve the uniformity of electric field distribution on the end tip of the therapeutic electrode 212 and the tip 90, thereby preventing spark discharge caused by uneven electric field distribution of the therapeutic electrode 212.

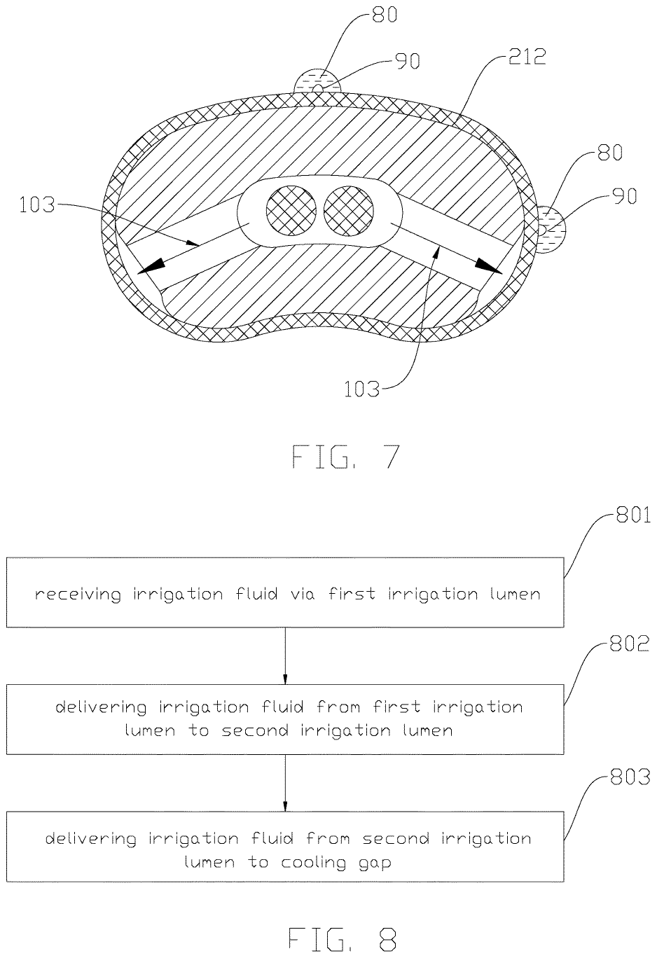

FIG. 8 illustrates an exemplary cooling method provided by the present disclosure, which can be applied to a pulsed electric field ablation catheter. The cooling method provided by at least one embodiment of the present disclosure may include the following steps:
Step 801: receiving an irrigation fluid via a first irrigation lumen.

Optionally, the irrigation fluid may be cold saline or other sterile liquids used for cooling, which is not limited herein. In the following description, cold saline is taken as an example for illustration.
Step 802: delivering the irrigation fluid from the first irrigation lumen to the second irrigation lumen.
Step 803: delivering the irrigation fluid from the second irrigation lumen to a cooling gap.

In one possible implementation, the pulsed electric field ablation catheter includes a catheter body 10 and an expandable portion 20. The catheter body 10 includes a main tube 11 and a push-pull tube 12. The push-pull tube 12 is slidably received in the main tube 11 and extends at least partially out of a distal end of the main tube 11.

The expandable portion 20 includes an expandable tube 21, and a plurality of branch tubes formed between a proximal end and a distal end of the expandable tube 21.

The proximal end of the expandable tube 21 is connected to the distal end of the main tube 11, and the distal end of the expandable tube 21 is connected to a distal end of the push-pull tube 12.

At least one therapeutic electrode 212 is disposed on the branch tube 211, and a cooling gap 30 is defined between the branch tube 211 and the therapeutic electrode 212, the cooling gap 30 being in communication with an external environment.

The main tube 11 is provided with a first irrigation lumen 111, and the branch tube 211 is provided with a second irrigation lumen 2111. The first irrigation lumen 111 is in fluid communication with the second irrigation lumen 2111, and the second irrigation lumen 2111 is in fluid communication with the cooling gap 30.

Since the first irrigation lumen 111 is provided in the main tube 11, the second irrigation lumen 2111 is provided in the branch tube 211, the first irrigation lumen 111 is in fluid communication with the second irrigation lumen 2111, and the second irrigation lumen 2111 is in fluid communication with the cooling gap 30, the irrigation fluid (such as cold saline 80) in the first irrigation lumen 111 can flow through the second irrigation lumen 2111 into the cooling gap 30 between the branch tube 211 and the therapeutic electrode 212 and then circulate to the external environment, thereby achieving cooling of the therapeutic electrode 212.

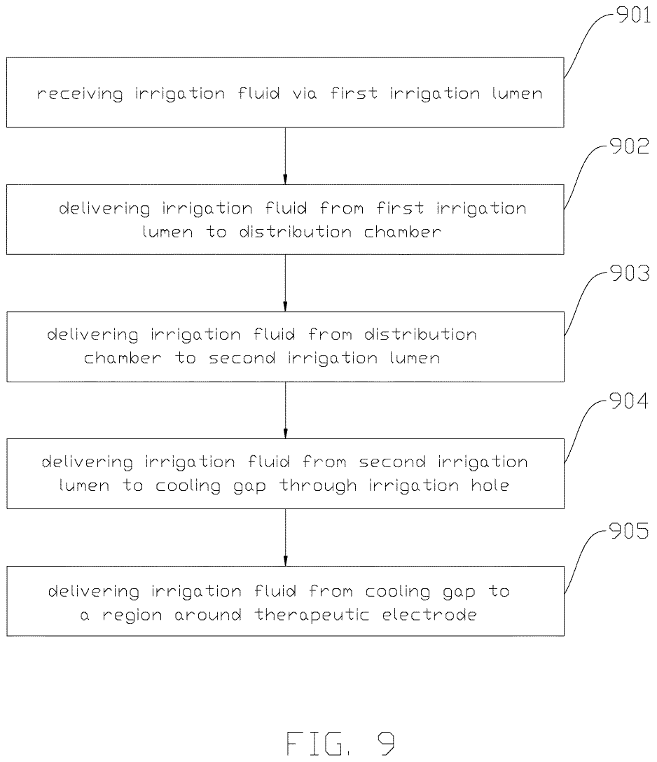

FIG. 9 illustrates another exemplary cooling method provided by the present disclosure, which may include:
Step 901: receiving an irrigation fluid via the first irrigation lumen.
Step 902: delivering the irrigation fluid from the first irrigation lumen to a distribution chamber.
Step 903: delivering the irrigation fluid from the distribution chamber to the second irrigation lumen.

The first irrigation lumen and the second irrigation lumen are in fluid communication with each other via the distribution chamber. The distribution chamber is defined by a gap between the proximal end of the expandable tube and the distal end of the main tube, together with an inner wall of the connecting tube. The connecting tube is configured to connect the expandable tube and the main tube.

Since the distribution chamber between the first irrigation lumen and the second irrigation lumen is in communication with at least one second irrigation lumen, the irrigation fluid in the first irrigation lumen can be uniformly distributed into the at least one second irrigation lumen.

In one possible implementation, the number of the first irrigation lumen is a first value, which can be set based on requirements and is not limited herein. By way of example, the number of the first irrigation lumens may be one.

Since an electrode is provided on each branch tube, each electrode requires a corresponding second irrigation lumen for cooling. However, the diameter of the main tube 11 is limited. As the number of irrigation lumens provided in the main tube 11 increases, the inner diameter of each individual irrigation lumen decreases, resulting in lower space utilization of the main tube 11. This, in turn, restricts the irrigation fluid flow in the irrigation lumen and compromises the cooling effect on the electrode. If only a single first irrigation lumen 111 is provided in the main tube 11 and is in fluid communication with a plurality of second irrigation lumens 2111 via the distribution chamber 40, there is no need to provide a corresponding plurality of irrigation lumens in the main tube 11. This achieves higher space utilization of the main tube 11 and effectively increases the cold saline irrigation flow rate of the pulsed electric field ablation catheter.
Step 904: delivering the irrigation fluid from the second irrigation lumen to the cooling gap through the irrigation hole.

The second irrigation lumen is in fluid communication with the cooling gap through the irrigation hole. The irrigation hole is provided in the branch tube at a position corresponding to the therapeutic electrode.

In one possible implementation, each branch tube is provided with at least one second irrigation lumen. Each second irrigation lumen on the branch tube corresponds to at least one irrigation hole.
Step 905: delivering the irrigation fluid from the cooling gap to a region around the therapeutic electrode.

When the pulsed electric field ablation catheter delivers cold saline 80 to cool the therapeutic electrode 212, the cold saline 80 is discharged to the vicinity of the therapeutic electrode 212 through the cooling gap 30, thereby increasing a contact area between the therapeutic electrode 212 and the blood 60. In addition, the cold saline 80 has a certain conductivity and an impedance generally lower than that of the blood 60. Thus, the cold saline 80 can mix with the blood 60 near the therapeutic electrode 212 to form a mixed conductive solution 70, which may help reduce a treatment impedance of the therapeutic electrode 212, allowing the pulsed electric field generated on the therapeutic electrode 212 to act on the target tissue more easily, thereby improving a treatment effect of the therapeutic electrode 212.

In one possible implementation, the irrigation fluid delivered to the region around the therapeutic electrode is used to envelop a tip on the therapeutic electrode.

In the case that a tip 90 is present on the therapeutic electrode, the irrigation fluid can envelop the tip 90 in the form of water droplet. This helps improve uniformity of an electric field distribution on an end face of the therapeutic electrode 212 and the tip 90, thereby preventing spark discharge caused by uneven electric field distribution of the therapeutic electrode 212.

The foregoing are merely preferred embodiments of the present disclosure and are not intended to limit the present disclosure. Any modifications, equivalent substitutions, and improvements made within the spirit and principle of the present disclosure shall be within the protection scope of the present disclosure.

What is claimed is:

1. A pulsed electric field ablation catheter comprising:
a catheter body comprising a main tube and a push-pull tube, the push-pull tube being slidably received in the main tube and extending at least partially out of a distal end of the main tube;
an expandable portion comprising an expandable tube and a plurality of branch tubes formed between a proximal and a distal end of the expandable tube, the proximal end of the expandable tube being connected to the distal end of the main tube, and the distal end of the expandable tube being connected to a distal end of the push-pull tube;
at least one therapeutic electrode disposed on each branch tube, and a cooling gap defined between the therapeutic electrode and the branch tube, the cooling gap being in communication with an external environment;
a first irrigation lumen provided in the main tube, and a second irrigation lumen provided in each branch tube, the first irrigation lumen being in fluid communication with the second irrigation lumen, and the second irrigation lumen being in fluid communication with the cooling gap.

2. The pulsed electric field ablation catheter according to claim 1, wherein the expandable tube is disposed around the push-pull tube, and the expandable portion further comprises a first sealing member and a second sealing member, the first sealing member disposed on the proximal end of the expandable tube and positioned between the expandable tube and the push-pull tube to seal a gap between the expandable tube and the push-pull tube;
wherein the first sealing member and the second sealing member each are elastic;
wherein the first sealing member comprises a sealing tube disposed in the expandable tube and around the push-pull tube, an outer wall of the sealing tube being fixedly connected to an inner wall of the expandable tube, and an inner wall of the sealing tube being in contact with an outer wall of the push-pull tube; and
wherein the expandable portion further comprises a second sealing member disposed on the distal end of the expandable tube for sealing a distal end of the second irrigation lumen.

3. The pulsed electric field ablation catheter according to claim 1, wherein the catheter body further comprises a connecting tube, two ends of the connecting tube being respectively disposed around or secured to the proximal end of the expandable tube and the distal end of the main tube to connect the expandable tube and the main tube.

4. The pulsed electric field ablation catheter according to claim 3, wherein a distribution chamber is defined by the proximal end of the expandable tube, the distal end of the main tube, and an inner wall of the connecting tube, and the first irrigation lumen and the second irrigation lumen are in fluid communication with each other via the distribution chamber.

5. The pulsed electric field ablation catheter according to claim 4, wherein the main tube is provided with a push-pull channel, the push-pull tube is axially slidable received in the push-pull channel, and the push-pull channel is in fluid communication with the distribution chamber.

6. The pulsed electric field ablation catheter according to claim 4, wherein a gap is provided between the proximal end of the expandable tube and the distal end of the main tube, such that the gap constitutes at least a portion of the distribution chamber.

7. The pulsed electric field ablation catheter according to claim 1, wherein an irrigation hole is provided in the branch tube at a position corresponding to the therapeutic electrode to place the second irrigation lumen in fluid communication with the cooling gap.

8. The pulsed electric field ablation catheter according to claim 7, wherein a cooling recess is provided on an outer wall of the branch tube at a position corresponding to the therapeutic electrode, the cooling gap is formed between the cooling recess and the therapeutic electrode, and the irrigation hole has one end in fluid communication with the cooling gap and another end in fluid communication with the second infusion channel.

9. The pulsed electric field ablation catheter according to claim 8, wherein the therapeutic electrode is an annular electrode disposed around the branch tube.

10. The pulsed electric field ablation catheter according to claim 1, wherein a plurality of cooling recesses and a plurality of irrigation holes are provided, the plurality of cooling recesses are circumferentially spaced apart along the branch tube, and the plurality of irrigation holes are in one-to-one correspondence with the plurality of cooling recesses.

11. The pulsed electric field ablation catheter according to claim 10, wherein two cooling recesses and two irrigation holes are provided, the two cooling recesses are respectively disposed on two opposite sides of the branch tube in a circumferential direction of the expandable tube, and the two irrigation holes respectively correspond to the two cooling recesses.

12. The pulsed electric field ablation catheter according to claim 1, wherein the at least one therapeutic electrode provided on the branch tube comprises a plurality of therapeutic electrodes spaced apart along a longitudinal direction of the branch tube;
wherein the plurality of therapeutic electrodes are spaced apart and disposed on a middle portion of the branch tube; and
wherein a distance between two adjacent therapeutic electrodes ranges from 1.5 mm to 3 mm.

13. The pulsed electric field ablation catheter according to claim 1, wherein the first irrigation lumen, the second irrigation lumen, and the cooling gap are capable of being filled with an irrigation liquid, and the irrigation fluid is capable of enveloping a tip on the therapeutic electrode.

14. A cooling method, applied to a pulsed electric field ablation catheter, the pulsed electric field ablation catheter comprising a catheter body and an expandable portion, the catheter body comprising a main tube and a push-pull tube slidably received in the main tube and extending at least partially out of a distal end of the main tube, the expandable portion comprising an expandable tube and a plurality of branch tubes formed between a proximal end and a distal end of the expandable tube, the proximal end of the expandable tube being connected to the distal end of the main tube, the distal end of the expandable tube being connected to a distal end of the push-pull tube, at least one therapeutic electrode disposed on each branch tube, a cooling gap defined between the therapeutic electrode and the branch tube and in communication with an external environment, a first irrigation lumen provided in the main tube, a second irrigation lumen provided in each branch tube, the first irrigation lumen being in fluid communication with the second irrigation lumen, and the second irrigation lumen being in fluid communication with the cooling gap;
the method comprising:
receiving an irrigation fluid via the first irrigation lumen;
delivering the irrigation fluid from the first irrigation channel to the second irrigation lumen; and
delivering the irrigation fluid from the second irrigation lumen to the cooling gap.

15. The cooling method according to claim 14, wherein the first irrigation lumen and the second irrigation lumen are in fluid communication with each other via a distribution chamber, the distribution chamber being defined by a gap between the proximal end of the expandable tube and the distal end of the main tube together with an inner wall of a connecting tube, the connecting tube being configured to connect the expandable tube and the main tube; and
the step of delivering the irrigation fluid from the first irrigation lumen to the second irrigation lumen comprises:
delivering the irrigation fluid from the first irrigation lumen to the distribution chamber; and
delivering the irrigation fluid from the distribution chamber to the second irrigation lumen.

16. The cooling method according to claim 14, wherein the second irrigation lumen is in fluid communication with the cooling gap through an irrigation hole provided in the branch tube at a position corresponding to the therapeutic electrode; and
the step of delivering the irrigation fluid from the second irrigation lumen to the cooling gap comprises:
delivering the irrigation fluid from the second irrigation lumen to the cooling gap through the irrigation hole.

17. The cooling method according to claim 14, wherein after delivering the irrigation fluid from the second irrigation lumen to the cooling gap, the method further comprises:
delivering the irrigation fluid from the cooling gap to a region around the therapeutic electrode.

18. The cooling method according to claim 17, wherein the irrigation fluid delivered to the region around the therapeutic electrode is configured to envelop a tip of the therapeutic electrode.

19. The method according to claim 14, wherein the number of the second irrigation lumen on each branch tube is at least one.

20. The method according to claim 14, wherein the number of the second irrigation lumen is a first value.
